(19) United States
(12) Patent Application Publication
(10) Pub. No.: US 2007/0179498 A1
(43) Pub. Date: Aug. 2, 2007

(54) ELECTROSURGERY ELECTRODE

(76) Inventor: Bruce MacDonald, Kitchener (CA)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/535,836

(22) Filed: Sep. 27, 2006

Related U.S. Application Data
(60) Provisional application No. 60/743,192, filed on Jan. 31, 2006.

(51) Int. Cl. A61B 18/14

(52) U.S. Cl. 606/45

(57) ABSTRACT

An electrosurgery electrode having a bent or angled portion which enables a physician to easily perform surgical procedures without his or her hand obstructing the view of the surgical site.

### CROSS-REFERENCE TO RELATED APPLICATIONS

This application claims the benefit of an earlier filed provisional application having Ser. No. 60/743,192, which is herein incorporated in its entirety.

### FIELD OF INVENTION

This invention relates generally to electrodes for use in electrosurgery procedures and, more particularly, to a bayonet shaped loop electrode for use in large loop excision of the transformation zone, an area containing abnormal cells including, namely, all cells that could become precancerous or develop into cervical cancer.

### BACKGROUND OF THE INVENTION

Cervical cancer is the third most common cause of cancer related deaths worldwide. In the United States, cervical cancer is the tenth leading cause of cancer deaths in women. The Papanicolaou smear (Pap Smear) is the standard method of screening for cervical cancer. An abnormal Pap Smear may include mild or slight cell changes, moderate cell changes, or severe cell changes. When moderate or severe cell changes are found, a physician will order a colposcopy.

Colposcopy is a diagnostic tool to determine the cause of abnormalities found in Pap smears. A colposcopy is a visual examination of the cervix using a colposcope, a large electric microscope. Acetic acid is placed on the cervix so that the cervical cells will absorb water and reduce their transparency. A bright light and colored filter on the end of the colposcope enable visual examination of the cervix and the highlighting of vascular patterns. If abnormalities are seen, biopsies of the cervical cells are obtained and sent to a pathologist to determine whether there is any displasia present.

Oftentimes, removal or destruction of the abnormal cells may occur at the same time a colposcopy is performed. Many procedures may be used to remove or destroy the abnormal cells including laser ablation, cold coagulation, cryotherapy, loop electrosurgical excision procedure (LEEP) and/or large loop excision of the transformation zone (LLETZ), cone biopsy, and hysterectomy. Laser ablation, cold coagulation, and cryotherapay treat only that part of the cervix containing abnormal cells. LEEP and/or LLETZ, cone biopsy and hysterectomy remove the whole area of the transformation zone including all cells that could become precancerous or develop into cervical cancer.

In LLETZ procedures, a local anesthetic is used for out patient treatment. A general anesthetic may be used if a very large area of tissue must be removed. The transformation zone that is removed during the LLETZ procedure is usually an area located inside the endocervical canal and therefore cannot always be seen clearly when a smear is taken. However, this area can be clearly seen during colposcopy which is why many physicians will prefer to perform LLETZ right away during colposcopy rather than have a patient return for such treatment.

During the LLETZ procedure, the transformation zone is cut away using a wire electrode tip formed in the shape of a loop and an electric current. The loop is used to scoop out the abnormal tissue in one piece and to seal any bleeding blood vessels. Since this procedure is performed using a speculum, and often done using a colposcope, the physician's hand is often in the way while trying to see and cut away the transformation zone. Most times, a physician will need to angle his or her hand to one side while cutting with existing electrodes used for the LLETZ procedure.

Accordingly, there is a need for an electrosurgery electrode that enables a physician to have a better line of sight to the cutting area in LLETZ procedures, as well as other surgical procedures where a physician's line of sight is obstructed, without the need to awkwardly position his or her hand to see where and how they are cutting with the electrode.

### SUMMARY OF THE INVENTION

This invention is directed to an electrosurgery electrode which enables a physician to easily perform surgical procedures without his or her hand obstructing the view of the surgical site. The electrosurgery electrode includes an electrode shaft having a first forked end, a second opposite end, and at least one bent or angled portion of the shaft located between the first forked end and the second opposite end, and a wire electrode tip coupled to the first forked end of the electrode shaft.

The first forked end of the electrode shaft includes a first fork and a second fork which both extend from the electrode shaft. The first and second forks may extend from the electrode shaft at various opposing angles with respect to the electrode shaft, or they may extend completely opposite one another (i.e. at opposite right angles with respect to the electrode shaft). The wire electrode tip has a first end and a second and they are connected to the first and second forks of the electrode shaft, respectively, thereby creating a loop having an opening therethrough. The loop may be of varying shapes and sizes and the wire used to form the loop may be of varying gauges. The second opposite end of the electrode shaft is connectable to an electrosurgery pencil which is powered by an electrosurgery unit.

With respect to one aspect of the invention, the distance between the first end of the wire electrode tip that is connected to the first fork of the electrode shaft and the second end of the wire electrode tip that is connected to the second fork of the electrode shaft, also know as the width of the electrode tip, is at least 5 millimeters. With respect to another aspect of the invention, the distance between the first forked end of the electrode shaft and the outermost point of the loop of the wire electrode tip, also known as the depth of the electrode tip, is at least 5 millimeters.

In one exemplary embodiment, the bent portion (which also defined to mean curved portion) or angled portion of the electrode shaft comprises a bayonet like shape. If the electrode shaft includes and angled portion, it may include at least two angles greater than ninety degrees, at least two angles equal to ninety degrees, or at least two angles less than ninety degrees.

In another exemplary embodiment, the wire electrode tip is connected to the forked end of the electrode shaft such that the wire electrode shift is positioned at an angle with respect to the plane of the forked end of the electrode shaft.

### BRIEF DESCRIPTION OF THE DRAWINGS

The present invention is hereafter described in conjunction with the following drawing figures, wherein like numerals denote like elements, and:

FIG. 1 is a top perspective view of a prior art electrode;

FIG. 2 is a top perspective view of an exemplary electrosurgery electrode according to the present invention;

FIG. 3 is a side elevational view of the electrosurgery electrode shown in FIG. 2;

FIG. 4 is a top perspective view of another exemplary embodiment of the electrosurgery electrode of the present invention;

FIG. 5 is a top perspective view of still another exemplary embodiment of the electrosurgery electrode of the present invention;

FIG. 6 is a top perspective view of yet another exemplary embodiment of the electrosurgery electrode of the present invention;

FIG. 7 is a side elevational view of the electrosurgery electrode shown in FIG. 6;

FIG. 8 is a top perspective view of another exemplary embodiment of the electrosurgery electrode of the present invention;

FIG. 9 is a side elevational view of the electrosurgery electrode shown in FIG. 8;

FIG. 10 is a top perspective view of yet another exemplary embodiment of the electrosurgery electrode of the present invention;

FIG. 11 is a top perspective view of still another exemplary embodiment of the electrosurgery electrode of the present invention;

FIG. 12 is a side elevational view of the electrosurgery electrode shown in FIG. 11;

FIG. 13 is a top perspective view of another exemplary embodiment of the electrosurgery electrode of the present invention;

FIG. 14 is a top perspective view of still another exemplary embodiment of the electrosurgery electrode of the present invention; and

FIG. 15 is a top perspective view of yet another exemplary embodiment of the electrosurgery electrode of the present invention

### DETAILED DESCRIPTION OF EXEMPLARY EMBODIMENTS

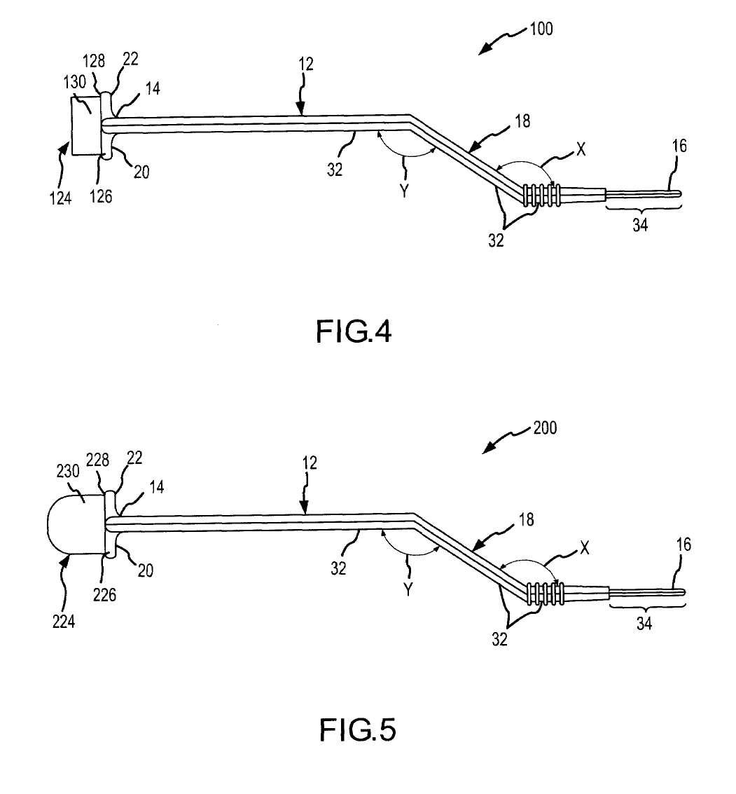

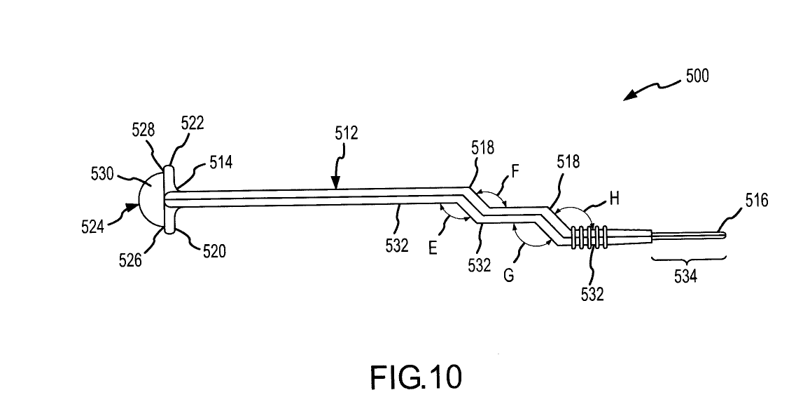

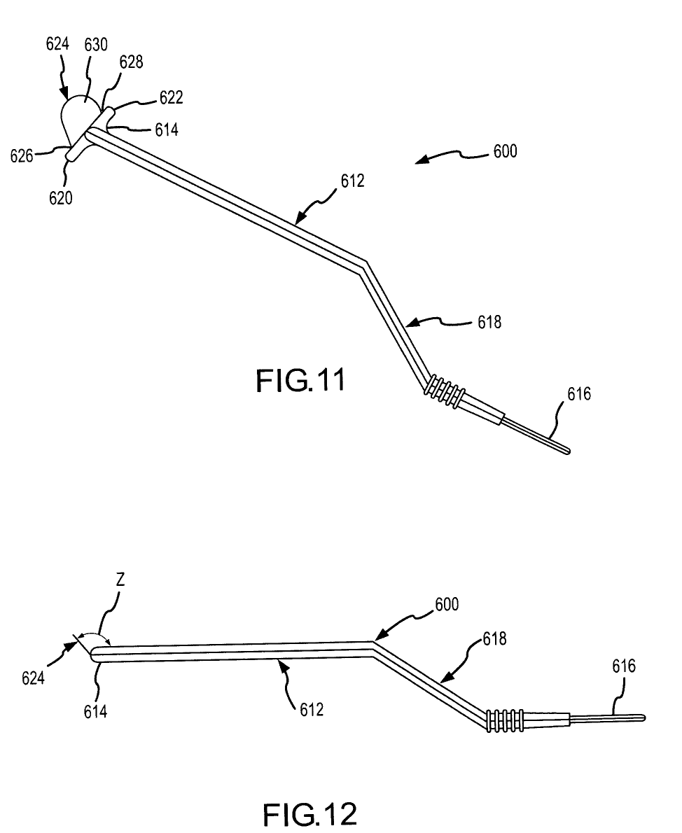

The following description is of exemplary embodiments of the invention only, and is not intended to limit the scope, applicability or configuration of the invention in any way. Rather, the following description is intended to provide a convenient illustration for implementing various embodiments of the invention. As will become apparent, various changes may be made in arrangement of the elements described in these embodiments without departing from the scope of the invention set forth in the appended claims. For example, in the context of the present invention, the apparatus hereof may include only those elements shown in FIG. 2 but may also include additional elements as those described with reference to FIGS. 10 and 11. Likewise, the same is true with the exemplary embodiments shown in FIGS. 4, 5, 6, and 8 in that these exemplary embodiments may also include additional elements as those described with reference to FIGS. 10 and 11. Furthermore, any of the exemplary embodiments shown and described may include a number of varying configurations, sizes, and gauges for the wire electrode tip.

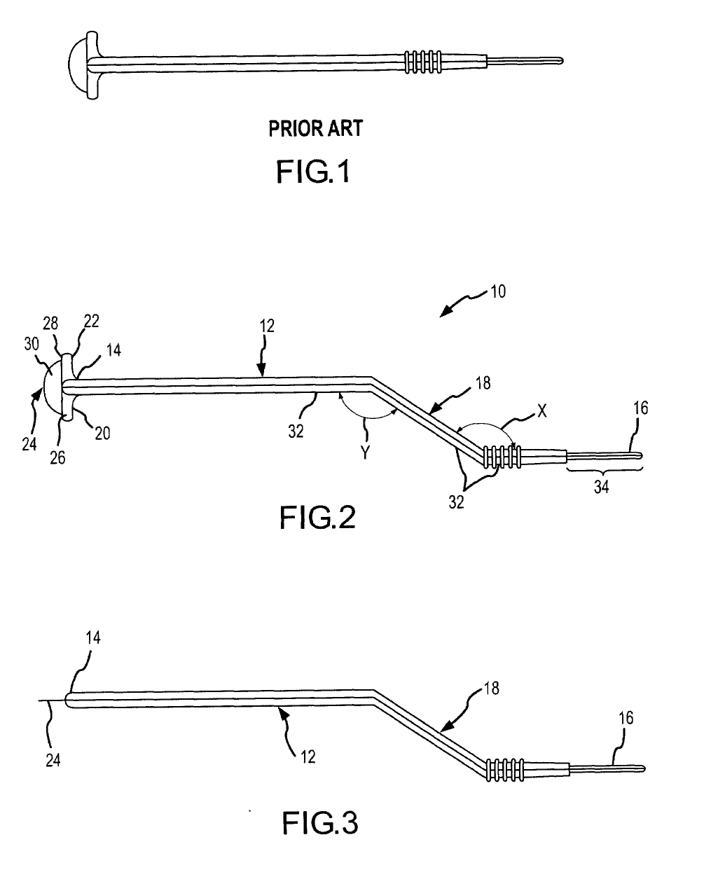

In general, the present invention provides an electrosurgery electrode having one or more bends, curves, or angles, which enables a physician to more easily perform surgical procedures without his or her hand obstructing the view of the surgical site. FIG. 1 shows a prior art electrosurgery electrode having a straight electrode shaft with first and second ends and a wire electrode tip coupled to one of the ends. An exemplary embodiment of the electrosurgery electrode of the present invention is illustrated in FIG. 2.

Electrosurgery electrode 10 shown in FIG. 2 includes an electrode shaft 12 having a first forked end 14 and a second end 16 located opposite forked end 14, and at least one angled portion 18 of electrode shaft 12 located between forked end 14 and second end 16.

First forked end 14 includes a first fork 20 and a second fork 22. Electrosurgery electrode 10 also includes a wire electrode tip 24 having a first end 26 connected to first fork 20 and a second end 28 connected to second fork 22 to form a loop having an opening 30 therethrough. Electrode shaft 12 is insulated with a non-conductive insulating material 32 except for a short exposed portion 34 near second end 16 of electrode shaft 12. Second end 16 of electrode shaft 12 is connectable to an electrosurgery pencil (not shown).

Electrode shaft 12 may be of varying lengths but angled portion 18 of electrode shaft 12 is preferably shorter in length than that portion of electrode shaft 12 located between angled portion 18 and first forked end 14 in order to provide more stability and accuracy to the movement of wire electrode tip 24 during electrosurgery. In the exemplary embodiment of electrosurgery electrode 10 shown in FIG. 2, angled portion 18 of electrode shaft 12 comprises a bayonet like shape having two angles (angle X and angle Y) measuring greater than ninety degrees. In addition, although first and second forks 20 and 22 are positioned opposite one another at ninety degree angles with respect to electrode shaft 12, first and second forks 20 and 22 may be positioned opposite one another at equal varying angles with respect to electrode shaft 12, i.e. they may be oppositely positioned at equal angles that are either greater than or less than ninety degrees with respect to electrode shaft 12.

FIG. 3 is a side elevation view of the exemplary electrosurgery electrode 10 illustrated in FIG. 2 showing electrode shaft 12 having first forked end 14, second exposed end 16, and angled portion 18, and wire electrode tip 24 coupled to first forked end 14 of electrode shaft 12. FIGS. 4 and 5 show additional exemplary embodiments of the electrosurgery electrode of the present invention. Electrosurgery electrodes 100 and 200 in FIGS. 4 and 5 show all of the same elements as electrosurgery electrode 10 shown in FIG. 1 with the exception of wire electrode tip 24. Electrosurgery electrode 100 instead includes wire electrode tip 124 having a first end 126 connected to first fork 20 and a second end 128 connected to second fork 22 to form a loop in the shape of a half square with opening 130. Electrosurgery electrode 200 instead includes wire electrode tip 224 having a first end 226 connected to first fork 20 and a second end 228 connected to second fork 22 to form having a loop in the shape of a half oval with opening 230.

It will be understood by those skilled in the art that the wire tip electrode of the present invention may comprise any number of configurations, sizes (including depth and width) and gauges. For example, the wire electrode tip may have a number of shapes including, but not limited to, circular, half circular, square, half square, rectangular, half rectangular, trapezoid, half trapezoid, hexagonal, half hexagonal, oval, and half oval, to name just a few. In addition, the width of the wire electrode tip (determined by measuring the longest length across the opening defined by the wire loop created from connecting the first and second ends of the electrode tip to the first and second forks of the electrode shaft) is preferably at least 5 millimeters and may be as much as 20 millimeters. The depth of the wire electrode tip (determined by measuring the distance between the first forked end of the electrode shaft and the outermost point of the loop of the wire electrode tip) is preferably at least 5 millimeters and may be as much as 15 millimeters. The electrode shaft may be comprised of any electrically conductive metal or material that is know to be used for, or could be used for, existing electrosurgery electrodes such as, for example, tungsten or stainless steel. Likewise, the insulating material which surrounds most of the electrode shaft may be comprise of any non-conductive material that is know to be used for, or that could be used for, existing electrosurgery electrodes. Furthermore, the electrode may have either a monopolar or bipolar design and may function as either a monopolar and/or bipolar electrode.

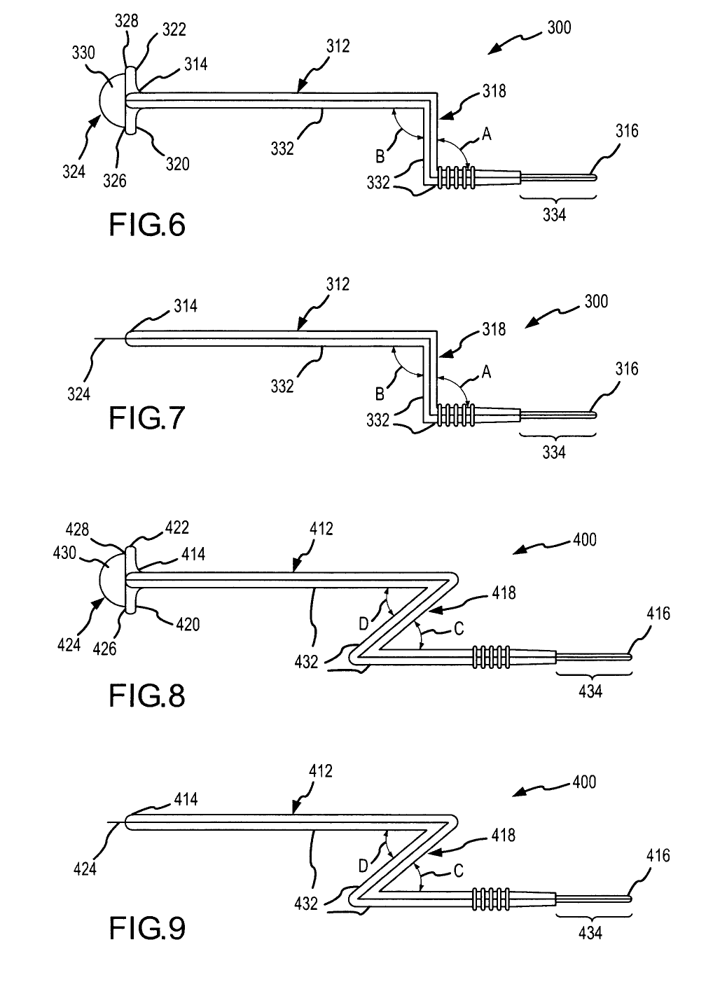

FIGS. 6 and 7 show a top perspective view and a side elevational view, respectively, of yet another exemplary embodiment of the electrosurgery electrode of the present invention. Electrosurgery electrode 300 shown in FIGS. 6 and 7 includes an electrode shaft 312 having a first forked end 314 and a second end 316 located opposite forked end 314, and at least one angled portion 318 of electrode shaft 312 located between forked end 314 and second end 316. First forked end 314 includes a first fork 320 and a second fork 322. Electrosurgery electrode 300 also includes a wire electrode tip 324 having a first end 326 connected to first fork 320 and a second end 328 connected to second fork 322 to form a loop having an opening 330 therethrough. Electrode shaft 312 is insulated with a non-conductive insulating material 332 except for a short exposed portion 334 near second end 316 of electrode shaft 312. Second end 316 of electrode shaft 312 is connectable to an electrosurgery pencil (not shown).

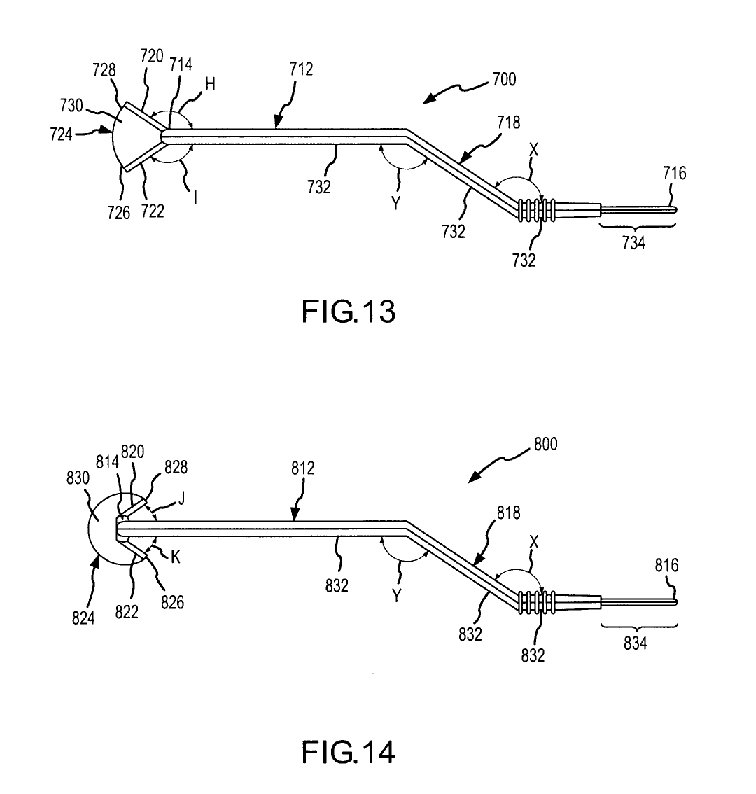

Electrode shaft 312 may be of varying lengths but angled portion 318 of electrode shaft 312 is preferably shorter in length than that portion of electrode shaft 312 located between angled portion 318 and first forked end 314 in order to provide more stability and accuracy to the movement of wire electrode tip 324 during electrosurgery. In the exemplary embodiment of electrosurgery electrode 300 shown in FIGS. 6 and 7, angled portion 318 of electrode shaft 312 comprises a step like shape having two right angles (angle A and angle B) measuring ninety degrees. In addition, although first and second forks 320 and 322 are positioned opposite one another at ninety degree angles with respect to electrode shaft 12, first and second forks 320 and 322 may be positioned opposite one another at equal varying angles with respect to electrode shaft 312, i.e. they may be oppositely positioned at equal angles that are either greater than or less than ninety degrees with respect to electrode shaft 312 (See, for example, FIGS. 13 and 14).

Turning now to FIGS. 8 and 9, a top perspective view and a side elevational view are shown, respectively, of yet another exemplary embodiment of the electrosurgery electrode of the present invention. Electrosurgery electrode 400 shown in FIGS. 8 and 9 includes an electrode shaft 412 having a first forked end 414 and a second end 416 located opposite forked end 414, and at least one angled portion 418 of electrode shaft 412 located between forked end 414 and second end 416. First forked end 414 includes a first fork 420 and a second fork 422. Electrosurgery electrode 400 also includes a wire electrode tip 424 having a first end 426 connected to first fork 420 and a second end 428 connected to second fork 422 to form a loop having an opening 430 therethrough. Electrode shaft 412 is insulated with a non-conductive insulating material 432 except for a short exposed portion 434 near second end 416 of electrode shaft 412. Second end 416 of electrode shaft 412 is connectable to an electrosurgery pencil (not shown).

Electrode shaft 412 may be of varying lengths but angled portion 418 of electrode shaft 412 is preferably shorter in length than that portion of electrode shaft 412 located between angled portion 418 and first forked end 414 in order to provide more stability and accuracy to the movement of wire electrode tip 424 during electrosurgery. In the exemplary embodiment of electrosurgery electrode 400 shown in FIGS. 8 and 9, angled portion 418 of electrode shaft 412 comprises a sharp bayonet like shape having two angles (angle C and angle D) measuring less than ninety degrees. In addition, although first and second forks 420 and 422 are positioned opposite one another at ninety degree angles with respect to electrode shaft 412, first and second forks 420 and 422 may be positioned opposite one another at equal varying angles with respect to electrode shaft 412, i.e. they may be oppositely positioned at equal angles that are either greater than or less than ninety degrees with respect to electrode shaft 412 (See, for example, FIGS. 13 and 14).

FIG. 10 is a top perspective view of yet another exemplary embodiment of the electrosurgery electrode 500 of the present invention. Electrosurgery electrode 500 includes an electrode shaft 512 having a first forked end 514 and a second end 516 located opposite forked end 514, and two angled portions 518 of electrode shaft 512 located between forked end 514 and second end 516. First forked end 514 includes a first fork 520 and a second fork 522. Electrosurgery electrode 500 also includes a wire electrode tip 524 having a first end 526 connected to first fork 520 and a second end 528 connected to second fork 522 to form a loop having an opening 530 therethrough. Electrode shaft 512 is insulated with a non-conductive insulating material 532 except for a short exposed portion 534 near second end 516 of electrode shaft 512. Second end 516 of electrode shaft 512 is connectable to an electrosurgery pencil (not shown).

Electrode shaft 512 may be of varying lengths but combined angled portions 518 of electrode shaft 512 is preferably shorter in length than that portion of electrode shaft 512 located between angled portions 518 and first forked end 514 in order to provide more stability and accuracy to the movement of wire electrode tip 524 during electrosurgery. In the exemplary embodiment of electrosurgery electrode 500 shown in FIG. 10, angled portions 518 of electrode shaft 512 comprise a staggered bayonet like shape having four angles (angle E, angle F, angle G and angle H) each measuring greater than ninety degrees. In addition, although first and second forks 520 and 522 are positioned opposite one another at ninety degree angles with respect to electrode shaft 512, first and second forks 520 and 522 may be positioned opposite one another at equal varying angles with respect to electrode shaft 512, i.e. they may be oppositely positioned at equal angles that are either greater than or less than ninety degrees with respect to electrode shaft 512.

FIGS. 11 and 12 show a top perspective view and a side elevational view, respectively, of yet another exemplary embodiment of the electrosurgery electrode 600 of the present invention. The exemplary embodiment shown in FIGS. 11 and 12 is the same as the exemplary embodiment shown in FIGS. 2 and 3 with the exception that wire electrode tip 624 is bent upward with respect to the plane of electrode shaft 612 located near first forked end 614 at an angle Z. Angle Z may comprise an angle that measures a variety of values including values that measure less than, equal to, or more than ninety degrees. In electrosurgery electrode 600, angle Z measures greater than ninety degrees with respect to electrode shaft 612.

FIGS. 13, 14, and 15 show top perspective views of additional exemplary embodiments of the electrosurgery electrode of the present invention. The exemplary embodiments shown in FIGS. 13, 14, and 14 are identical to the exemplary embodiment shown in FIG. 2 with the exception of the wire electrode tip 24. In the exemplary embodiment shown in FIG. 13, electrosurgery electrode 700 includes an electrode shaft 712 having a first forked end 714 and a second end 716 located opposite forked end 714, and at least one angled portion 718 of electrode shaft 712 located between forked end 714 and second end 716. First forked end 714 includes a first fork 720 and a second fork 722 where the first and second forks 720 and 722 are positioned opposite one another at equal angles with respect to electrode shaft 712, where the equal angles (angles H and I) are greater than ninety degrees with respect to electrode shaft 712. Electrosurgery electrode 700 also includes a wire electrode tip 724 having a first end 726 connected to first fork 720 and a second end 728 connected to second fork 722 to form a loop having an opening 730 therethrough. Electrode shaft 712 is insulated with a non-conductive insulating material 732 except for a short exposed portion 734 near second end 716 of electrode shaft 712. Second end 716 of electrode shaft 712 is connectable to an electrosurgery pencil (not shown).

In the exemplary embodiment shown in FIG. 14, electrosurgery electrode 800 includes an electrode shaft 812 having a first forked end 814 and a second end 816 located opposite forked end 814, and at least one angled portion 818 of electrode shaft 812 located between forked end 814 and second end 816. First forked end 814 includes a first fork 820 and a second fork 822 where the first and second forks 820 and 822 are positioned opposite one another at equal angles with respect to electrode shaft 812, where the equal angles (angles J and K) are less than ninety degrees with respect to electrode shaft 812. Electrosurgery electrode 800 also includes a wire electrode tip 824 having a first end 826 connected to first fork 820 and a second end 828 connected to second fork 822 to form a loop having an opening 830 therethrough. Electrode shaft 812 is insulated with a non-conductive insulating material 832 except for a short exposed portion 834 near second end 816 of electrode shaft 812. Second end 816 of electrode shaft 812 is connectable to an electrosurgery pencil (not shown).

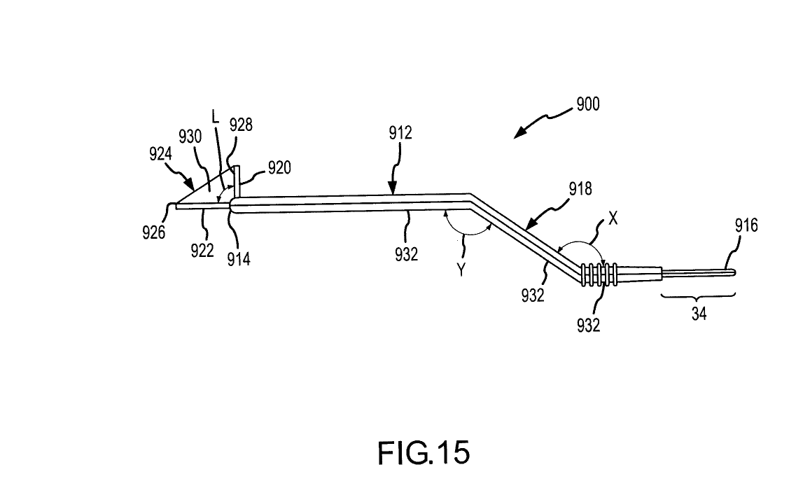

Finally, in the exemplary embodiment shown in FIG. 15, electrosurgery electrode 900 includes an electrode shaft 912 having a first forked end 914 and a second end 916 located opposite forked end 914, and at least one angled portion 918 of electrode shaft 912 located between forked end 914 and second end 916. First forked end 914 includes a first fork 920 and a second fork 922 where the first and second forks 920 and 922 are positioned at a ninety degree angle (angle L) with respect to one another. Electrosurgery electrode 900 also includes a wire electrode tip 924 having a first end 926 connected to first fork 920 and a second end 928 connected to second fork 922 to form a loop having an opening 930 therethrough. Electrode shaft 912 is insulated with a non-conductive insulating material 932 except for a short exposed portion 934 near second end 916 of electrode shaft 912. Second end 916 of electrode shaft 912 is connectable to an electrosurgery pencil (not shown).

The foregoing description is of exemplary embodiments of the subject invention. It will be appreciated that the foregoing description is not intended to be limiting; rather, the exemplary embodiments set forth herein merely set forth some exemplary applications of the subject invention. It will be appreciated that various changes, deletions, and additions may be made to the components and steps discussed herein without departing from the scope of the invention as set forth in the appended claims.

1. An electrosurgery electrode comprising:
an insulated electrode shaft having a first forked end comprising a first fork and a second fork, a second end opposite the first forked end, and at least one angled portion located between the first forked end and the second opposite end; and
a wire electrode tip comprising a first end and a second end connected to the first and second forks of the first forked end of the insulated electrode shaft, respectively, thereby creating a loop having an opening therethrough.

2. The electrosurgery electrode of claim 1 wherein the second opposite end of the insulated electrode shaft is connectable to an electrosurgery pencil.

3. The electrosurgery electrode of claim 1 wherein the wire electrode tip comprises a generally half circular shape.

4. The electrosurgery electrode of claim 1 wherein the wire electrode tip comprises a generally half square shape.

5. The electrosurgery electrode of claim 1 wherein the electrode tip comprises a generally half oval shape.

6. The electrosurgery electrode of claim 1 wherein the distance between the first end of the electrode tip connected to the first fork and the second end of the electrode tip connected to the second fork is at least 5 millimeters.

7. The electrosurgery electrode of claim 1 wherein the distance between the first forked end of the insulated electrode shaft and the outermost point of the loop of the electrode tip is at least 5 millimeters.

8. The electrosurgery electrode of claim 1 wherein the insulated electrode shaft comprises a bayonet like shape.

9. The electrosurgery electrode of claim 1 wherein the at least one angled portion of the insulated electrode shaft comprises at least two angles greater then 90 degrees.

10. The electrosurgery electrode of claim 1 wherein the at least one angled portion of the insulated electrode shaft comprises at least two 90 degree angles.

11. The electrosurgery electrode of claim 1 wherein the wire electrode tip is connected to the forked end of the insulated electrode shaft such that the wire electrode tip is positioned at an angle with respect to the plane of the forked end of the insulated electrode shaft.

12. The electrosurgery electrode of claim 1 wherein the first and second forks are opposite one another and form ninety degree angles with respect to the insulated electrode shaft.

13. The electrosurgery electrode of claim 1 wherein the second opposite end of the insulated electrode shaft includes an exposed portion of conductive electrode.

14. An electrosurgery electrode for use in large loop excision of the transformation zone comprising:
An insulated electrode shaft having a first forked end comprising a first fork and a second fork, a second end opposite the first forked end for coupling to an electrosurgery pencil, and at least one bent portion located between the first forked end and the second opposite end; and
a wire electrode tip comprising a first end and a second end connected to the first and second forks of the first forked end of the insulated electrode shaft, respectively, thereby creating a loop having an opening therethrough.

15. The electrosurgery electrode of claim 14 wherein the insulated electrode shaft comprises a bayonet like shape.

16. The electrosurgery electrode of claim 14 wherein the wire electrode tip comprises a generally half circular shape.

17. The electrosurgery electrode of claim 14 wherein the wire electrode tip comprises a generally half square shape.

18. The electrosurgery electrode of claim 14 wherein the wire electrode tip comprises a generally half oval shape.

19. The electrosurgery electrode of claim 14 wherein the distance between the first end of the electrode tip connected to the first fork and the second end of the electrode tip connected to the second fork is at least 5 millimeters.

20. The electrosurgery electrode of claim 14 wherein the distance between the first forked end of the insulated electrode shaft and the outermost point of the loop of the electrode tip is at least 5 millimeters.

21. The electrosurgery electrode of claim 14 wherein the wire electrode tip is connected to the forked end of the insulated electrode shaft such that the wire electrode tip is positioned at an angle with respect to the plane of the forked end of the insulated electrode shaft.

22. The electrosurgery electrode of claim 14 wherein the first and second forks are opposite one another and form ninety degree angles with respect to the insulated electrode shaft.

23. The electrosurgery electrode of claim 14 wherein the second opposite end of the insulated electrode shaft includes an exposed portion of conductive electrode.
